(19) United States
(12) Patent Application Publication
(10) Pub. No.: US 2013/0289531 A1
(43) Pub. Date: Oct. 31, 2013

(54) LUBRICIOUS MEDICAL TUBING

(71) Applicant: CORDIS CORPORATION, Bridgewater, NJ (US)

(72) Inventors: Marcos PAGAN, El Paso, TX (US); Patricia A. DAVIS-LEMESSY, San Jose, CA (US); Armando A. GRAUPERA, Miami, FL (US)

(21) Appl. No.: 13/801,527

(22) Filed: Mar. 13, 2013

Related U.S. Application Data
(60) Provisional application No. 61/618,764, filed on Mar. 31, 2012.
(60) Provisional application No. 61/666,846, filed on Jun. 30, 2012.

(51) Int. Cl. C08L 27/18; C08L 77/00; A61M 25/00

(52) U.S. Cl. CPC C08L 27/18; A61M 25/00; C08L 77/00
USPC 604/523; 525/190; 525/93; 524/502; 428/36.9

(57) ABSTRACT

A composition of a polyamide and PTFE produces a lubricious surface on extruded medical tubing. The small size of the PTFE powder when compounded with polyamide disperses uniformly and produces an intrinsically lubricious polymer. Such a composition can be useful in medical, intralumenal tubing.

### RELATED APPLICATIONS

This application is a non-provisional application of U.S. Provisional Application Ser. No. 61/618,764, filed Mar. 31, 2012, currently pending and U.S. Provisional Application Ser. No. 61/666,846, filed Jun. 30, 2012, currently pending, both of which are incorporated by reference in their entirety herein.

### BACKGROUND

1. Technical Field

The invention relates to the field of medical, intralumenal tubing.

2. Related Devices and Methods

Many medical procedures use tubing. In particular, the tubing is advanced in body lumens, and is thus characterized generally as medical, intralumenal tubing.

It is desirable at times to reduce the friction between the medical, intralumenal tubing and the walls of the body lumen, or between the medical tubing and other medical devices expected to have contact and move against it. Examples of the contact between the medical tubing and the body lumen are esophageal balloon catheters and the esophagus, sinuplasty catheters and the sinus cavity, and the vasculature and any of the many catheters used in procedures in the vasculature, or accessed through the vasculature: e.g., angioplasty catheters, ablation catheters, guiding catheters, diagnostics catheters, stent delivery systems, implant delivery systems, etc. In vascular procedures oft times several catheters are introduced through a vessel, and often one catheter inside another. Examples of such are guiding catheters with guide wires and/or imaging devices running through them, or angioplasty catheters, positioning and measurement devices. In these cases it is the inner surface of the guiding catheter and the outer surface of the other device that would desirably have insignificant kinetic friction when in moving contact with one another (e.g., rotational or longitudinal) or static friction to overcome before moving, once in contact.

Solutions to reducing friction include using intrinsically lubricious polymers or adding a layer of lubricious material, such a coating. Polytetrafluoroethylene (PTFE) and high density polyethylene (HDPE) have been used as intrinsically lubricious polymers for medical tubing. And coatings, such as those based on PVP or hyaluronic acid, have been applied to polymers that are not sufficiently intrinsically lubricious for medical, intralumenal tubing. Coatings wear, and PTFE and HDPE do not have the properties on their own needed to resist torsion or sufficient strength as is desirable for the medical procedures. Thus, past solutions have included making medical tubing out of two polymers, coaxially situated with one another. In the cases of guide catheters, the innermost layer of the tubing is made from PTFE or HDPE and the other coaxial (not innermost) layer is made from a polyamide, either a homopolyamide, such as Nylon™, or a copolymer, such as a polyetheramide, including brands such as PEBAX®. Even then, these inner and outer layers have not bonded well together, often requiring an intermediate, at least third, layer which acts as an adhesive. Of course, the more layers one uses in medical tubing, the greater the wall thickness typically is, which may make the outer diameter larger than desired for a given inner diameter, or a smaller inner diameter than desired for a given outer diameter.

### BRIEF DESCRIPTION OF THE FIGURES

The figures are merely exemplary and are not meant to limit the present invention.

FIG. 1 is a chart comparing the static COFs of extrusions of two different NFPBs with an extrusion of 100% PTFE.

FIG. 2 is another chart comparing the kinetic COFs of extrusions of two different NFPBs with an extrusion of 100% PTFE.

FIG. 3 is a box plot of Static Median COF(S) of Hydrophobic and Hydrophilic Coatings on Nylon Surface.

FIG. 4 is a box plot of Kinetic Median COF of Hydrophobic and Hydrophilic Coatings on Nylon Surface.

FIG. 5 is a drawing of a front perspective view of the friction tester used to generate the results in FIGS. 1-4.

FIGS. 6A and 6B illustrate the sled recommended for use with the ASTM D 1894-08 standard.

FIGS. 7A, 7B, and 7C illustrate the “ContraForm” Sled. The assembled sled is shown in FIG. 7A. FIG. 7B is a perspective view of the sled base, which is rounded. FIG. 7C is a dimensioned schematic of the assembled sled.

FIG. 8 is top view of several standard shape guide catheters.

FIG. 9 is perspective view of an angioplasty catheter.

### DETAILED DESCRIPTION

The terms “tube” and “tubular” are used in their broadest sense, to encompass any structure arranged at a radial distance around a longitudinal axis. Accordingly, the terms “tube” and “tubular” include any structure that (i) is cylindrical or not, such as for example an elliptical or polygonal cross-section, or any other regular or irregular cross-section; (ii) has a different or changing cross-section along its length; (iii) is arranged around a straight, curving, bent or discontinuous longitudinal axis; (iv) has an imperforate surface, or a periodic or other perforate, irregular or gapped surface or cross-section; (v) is spaced uniformly or irregularly, including being spaced varying radial distances from the longitudinal axis; or (vi) has any desired combination of length or cross-sectional size.

In the following descriptions of compositions, given percentages reflect percentage of the total weight of the composition.

“Lubricant” refers to an additive that imparts lubricity to the extruded component. It does not include additives to the ingredients for the purposes of processing in the compounding screw, sometimes referred to as an internal lubricant or a dispersion aid. For the avoidance of confusion, “dispersion aid” refers to the additive that allows for optimal mixing of the ingredients that make up the compound.

Embodiments of the invention are extruded films or tubes made from a polymer composition that includes PTFE particles which are dispersed in a carrier polymer with which they are immiscible. Improved lubricity compared to 100% PTFE is seen in tests of extrusions of compositions that include only 10% PTFE. In one composition with improved lubricity over 100% PTFE, the PTFE powder has a mean particle size in the range of 10 to 60 microns (micrometers). In another composition with improved lubricity over 100% PTFE, the PTFE powder has a mean particle size in the range of 200-700 nanometers. In some embodiments, the composition is 10% by weight PTFE powder compounded with 90% by weight nylon, or other polyamide based polymer. It is expected that improved lubricity compared to 100% PTFE will be present in extrusions made from compositions with as little as 1% PTFE powder through 25% PTFE powder, if the mean size of the powder is between 200 and 700 nanometers. In particular, it is expected that improved lubricity compared to 100% PTFE will be present in extrusions made with 5%, 15%, 20%, and 25% PTFE powder, if the mean size of the powder is between 200 and 700 nm. In some embodiments, the PTFE has a mean particle size between 200-700 nanometers and dry agglomerates in the range of 10-15 microns.

The polymer in which the PTFE particles are dispersed can be a homopolymer (for e.g., a polyamide homopolymer or a polyester homopolymer), a co-polymer such as a polyetheramide, or HYTREL® polyurethanes, or blends of the above.

Examples of the polyamide homopolymers are polymers sold as Grilamid® L series of nylon 12 polymers, Grilamid L series of nylon 12 polymers, nylon 11 homopolymers, nylon 1010, nylon 1012, nylon 6,6; and/or nylon 6 polymers. The carrier polymer can also consist of blends of homopolymer polyamides.

Examples of copolymer polyamides such as polyetheramides, sometimes known as polyether block amide (PEBA), are polymers sold as Pebax® series, Grilflex®, or other nylon 6-, nylon 11-, or nylon 12-based PEBA.

Other polymers can be used along with or instead of each polyamide component.

They are polymers such as poly(meth)acrylates, vinyl polymers, polyolefins, halogenated polymers, polymers having urethane groups, polybutyals, nylon, silicones, polycarbonate, or polysulfone.

Nylon Fluoro Polymer Blends (“NFPBs”)

Improved lubricity is obtained by blending submicron-sized PTFE particles having lowered surface energy along with other lubricating additives into nylon and PEBA resins. Both dry and wet tested lubricity enhancements can be realized for the NFPBs. Blending submicron-sized PFTE particles resolves this non-solubility issue, thus providing a compound with well dispersed PTFE. Once the article is made, by extrusion or other process, the dispersed PTFE particulates bloom to the surface of the item and impart the lubricious properties of PTFE to the article.

For example, nylon 12 homopolymer and different grades of compatible thermoplastic elastomers (TPE) also known as polyether block amide (PEBA) polymers can be blended with submicron-sized PTFE powder to create NFPBs. When the submicron-sized PTFE is compounded into the nylon resins, the resulting extrusion surface is more lubricious than either a nylon surface or a PTFE surface, such that the tested coefficient of friction is improved compared a surface without the submicron-sized additive.

The nature of this blend, in which a homopolymer and a PEBA polymer are combined with PTFE particles, allows for percentage adjustments such that the durometer of the blend can be customized by the selection of the homopolymer and PEBA grades. For example, the formulation of the blend is adjustable such that the ratio of the amounts of the first and second ingredients shift from as high as 17 (Table 1) to as low as 0.058 (Table 2).

Moreover, the selected grades of the polymers are changeable so that the durometer of the NFPB compound can be customized, as needed. Compared to the example in Table 3, substituting a lower durometer (40D) Vestamid PEBA for the second ingredient results in the formulation as seen in Table 4.

Furthermore, the amount of fluoropolymer additive can range from 1-25%. A preferred fluoropolymer additive is Shamrock Technologies NanoFLON® P 39B Thermoplastic Grade PTFE Additive.

### EXAMPLES

An embodiment of a NFPB consists of the following ingredients in Table 1 below; however, the ranges of the ingredients can range widely as needed (see Table 2). In addition, the durometer of the NFPB results from the durometers of the selected homopolymer and PEBA ingredients (see Table 4).

TABLE 1
NFPB Ingredients for Enhanced Lubricity - Example 1

| | Percent by |
|---|---|
| Ingredient | Weight (%) |
| 1) Nylon 12 Vestamid L2101F or L2140 (homopolymer), or | 85.0 |
| equivalent | |
| 2) Nylon 12 Vestamid E62-S3 (PEBA), or equivalent | 5.0 |
| 3) Submicron-sized PTFE powder, or equivalent | 10.0 |

TABLE 2
NFPB Ingredients for Enhanced Lubricity - Example 2

| | Percent by |
|---|---|
| Ingredient | Weight (%) |
| 1) Nylon 12 Vestamid L2101F or L2140 (homopolymer), or | 5.0 |
| equivalent | |
| 2) Nylon 12 Vestamid E62-S3 (PEBA), or equivalent | 85.0 |
| 3) Submicron-sized PTFE powder, or equivalent | 10.0 |

TABLE 3
NFPB Ingredients for Enhanced Lubricity and Selected Durometer -
Example 3

| | Percent |
|---|---|
| Ingredient | by Weight (%) |
| 1) Nylon 12 Vestamid L2101F or L2140, or equivalent | 52.0 |
| 2) Nylon 12 Vestamid E62-S3, or equivalent | 38.0 |
| 3) Submicron-sized PTFE powder, or equivalent | 10.0 |

TABLE 4
NFPB Ingredients for Enhanced Lubricity and Selected Lower
Durometer - Example 4

| | Percent |
|---|---|
| Ingredient | by Weight (%) |
| 1) Nylon 12 Vestamid L2101F or L2140, or equivalent | 52.0 |
| 2) Nylon 12 Vestamid E40-S3, or equivalent | 38.0 |
| 3) Submicron-sized PTFE powder, or equivalent | 10.0 |

In some embodiments, an internal lubricant or dispersion aid or dispersant may be necessary or advantageous depending on the non-fluoro-additive ingredients (in the above examples, the nylon and the PEBA) to ensure proper mixing of the ingredients to provide a consistent character to the resulting blend. Examples of such dispersion aids include zinc stearate, calcium stearate, sodium stearate, and magnesium stearate.

In some embodiments, no dispersant or dispersal aid is used in compounding the ingredients and the PTFE will sufficiently disperse to provide improved lubricity and a quality extrusion. However, the greater the mean particle size of the PTFE particles, the more likely agglomeration may occur that may be undesirable. If larger PTFE particles agglomerate, which agglomeration has a larger dimension than the radial thickness of the extrusion, the surface is textured, or bubbled. In some instances, the polymer covering the agglomeration of PTFE particles is thin enough to break and expose the particles which fall out as powder. The agglomerations are believed to be due to the difference in solubility of PTFE and the polyamide polymer.

Lubricity

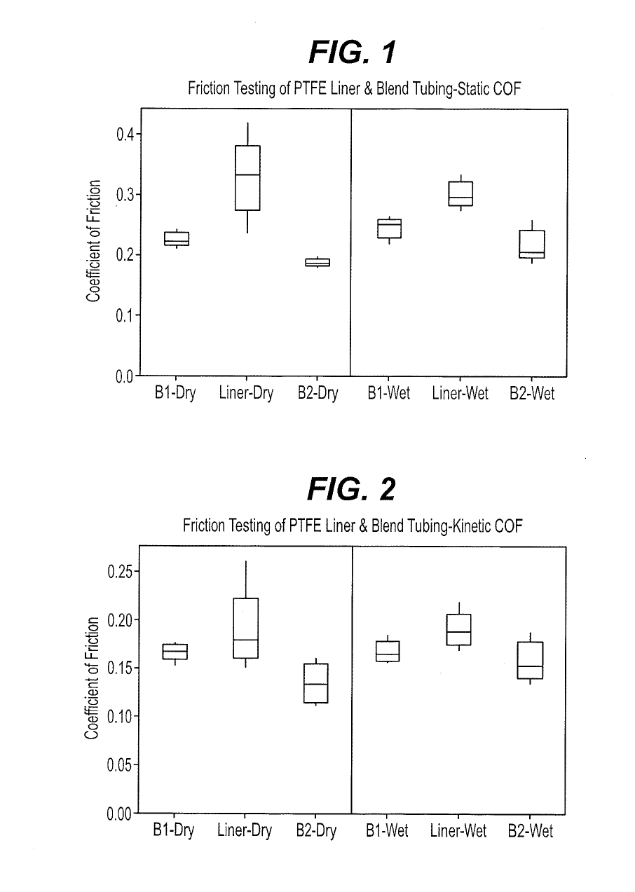

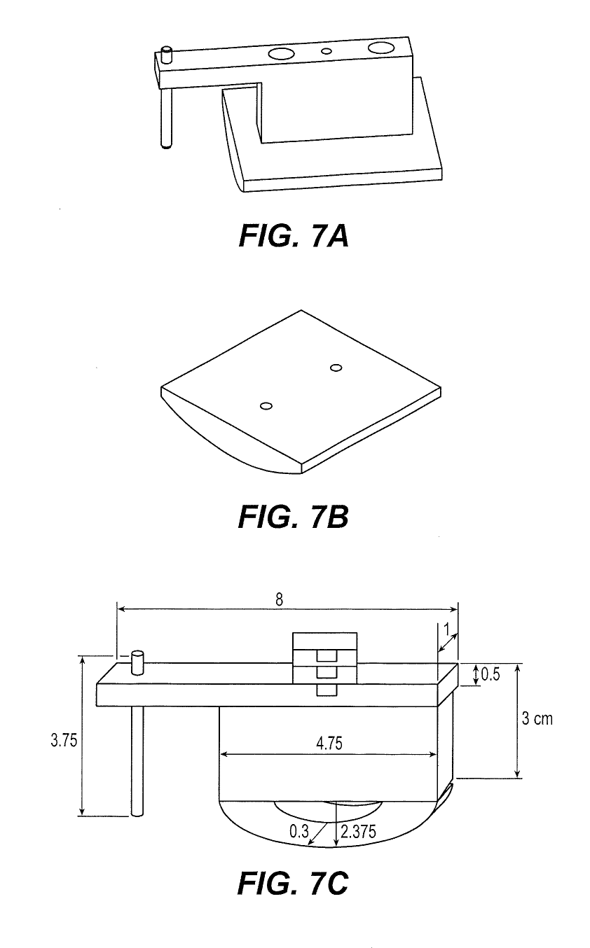

Increased lubricity, or lubricity enhancement, is inversely related to the coefficient of friction. FIGS. 1 and 2 display the static and kinetic coefficients-of-friction (COFs), respectively, of a guiding catheter with various liners. The one guide catheter has a 100% PTFE liner, one has NFPB B 1, which is a blend of fluoropolymer particles in nylon polymers, where the PTFE particles are not specified as sub micron mean particle size, and one has NFPB B2, which is a blend according to Example 3 (Table 3), which exhibits the lowest static and kinetic COFs among all the samples when tested dry and wet. The samples were prepared according to ASTM D 1894-08 (Slip and Friction Test Procedure) with modifications as described below. All samples were tested in both the dry and wet states on the friction tester shown in FIG. 7.

FIGS. 1 & 2 display the coefficients-of-friction (COFs) of a guide catheter's 100% PTFE liner and two embodiments, B1 and B2, of the composition, labeled as a “NFPBs”. The second embodiment (B2), displays the lowest static and kinetic COFs among all the samples when tested dry and wet.

Improved Lubricity: Additional Additives

Lubricating additives may also be added to the NFPBs. In some embodiments, the percentage by weight of the PTFE powder stays the same, and the percentage of polyamide polymer (whether solely nylon (homopolymer) or solely PEBA or a blend of the two) decreases to accommodate the added lubricant. In some embodiments, the percentage of the PTFE powder can be reduced along with the percentage of polyamide polymer to accommodate the added lubricant.

The introduction of additional lubricious additives such as carnauba, silicone or hydrophilic entities into the compound further improves the lubricity of the resulting extrusions. Examples of hydrophilic additives are poly vinyl alcohol (PVOH), polyethylene oxide (Polyox), and polyethylene glycol (PEG). As the additives bloom to the surfaces of the extruded component they provide a lubricious surface.

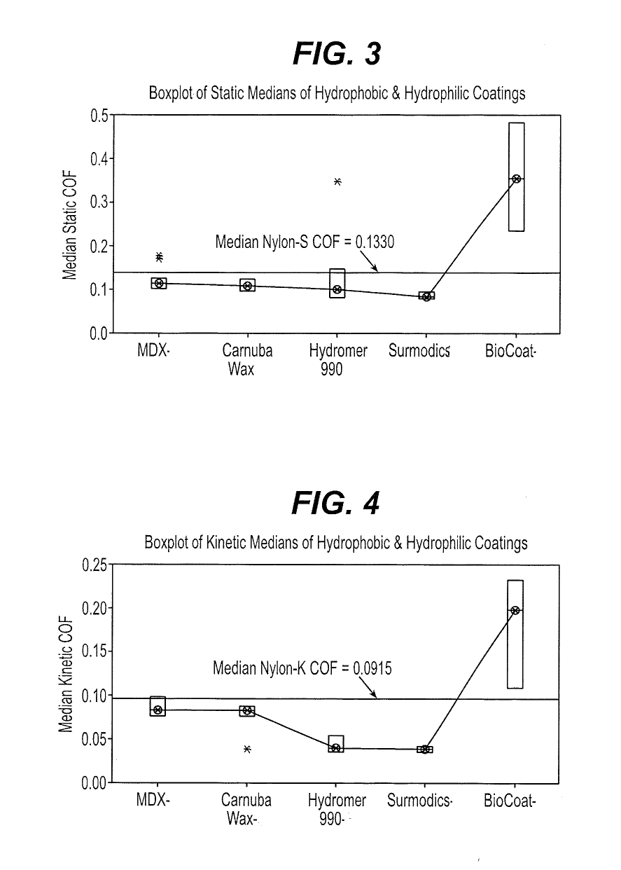

Lubricious additives display lower static and kinetic COFs compared to those of nylon. Additives such as MDX, a silicone oil, such as MDX 4-4159 Fluid, or carnauba wax; as well as other hydrophilic agents, such as PVOH (Table 6), or Hydromer® 990 or Surmodics® lubricant to further enhance the lubricity of the NFPB. FIGS. 3 & 4 display box plots of in vitro measured wet static and kinetic COFs of different hydrophobic and hydrophilic coatings. These additives can be incorporated into the NFPB at the time of initial compounding or in a separate downstream process, such as, e.g., coating by dipping or spraying, among others. Based on the box plots, two hydrophilic additives impart the lowest static and kinetic COFs.

Potential hydrophilic additives include polyalkylene glycols, hyaluronic acid, chondroitan sulfate, chitosan, glucosaminoglucans, dextran, dextrin, dextran sulfate, cellulose acetate, carboxymethyl cellulose, hydroxyethyl cellulose, cellulosics, polypeptides, poly(2-hydroxyethyl methacrylate), polyacrylamide, polyacrylimide, poly(ethylene amine), poly(allyl amine), poly(vinyl pyrrolidone), poly(vinyl alcohol), poly(acrylic acid), poly(methacrylic acid), acrylic acid copolymers, methacrylic acid copolymers, polyvinyl alkyl ethers, non-ionic tetrafunctional block-copolymer surfactants, gelatin, collagen, albumin, chitin, heparin, elastin, fibrin, Irgasurf® HL 560.

TABLE 5
NFPB Ingredients for Enhanced Hydrophobic Lubricity - Example 5

| Ingredient | Percent by Weight (%) |
|---|---|
| 1) Nylon 12 Vestamid L2101F | 52.0 |
| 2) Nylon 12 Vestamid E62-S3 | 37.8 |
| 3) Submicron-sized PTFE powder, or equivalent | 10.0 |
| 4) Lubricant (PolyOx), or equivalent) | 0.20 |

TABLE 6
Similar Durometer NFPB Ingredients for Enhance Hydrophilic Lubricity -
Ex. 6

| Part Description | Percent by Weight (%) |
|---|---|
| 1) Nylon 12 Vestamid L2101F Natural | 51.0 |
| 2) Nylon 12 Vestamid E62-S3 Natural | 37.0 |
| 3) Submicron-sized PTFE powder, or equivalent | 10.0 |
| 4) Hydrophilic lubricant (PVOH or equivalent) | 2.0 |

Friction Testing Method

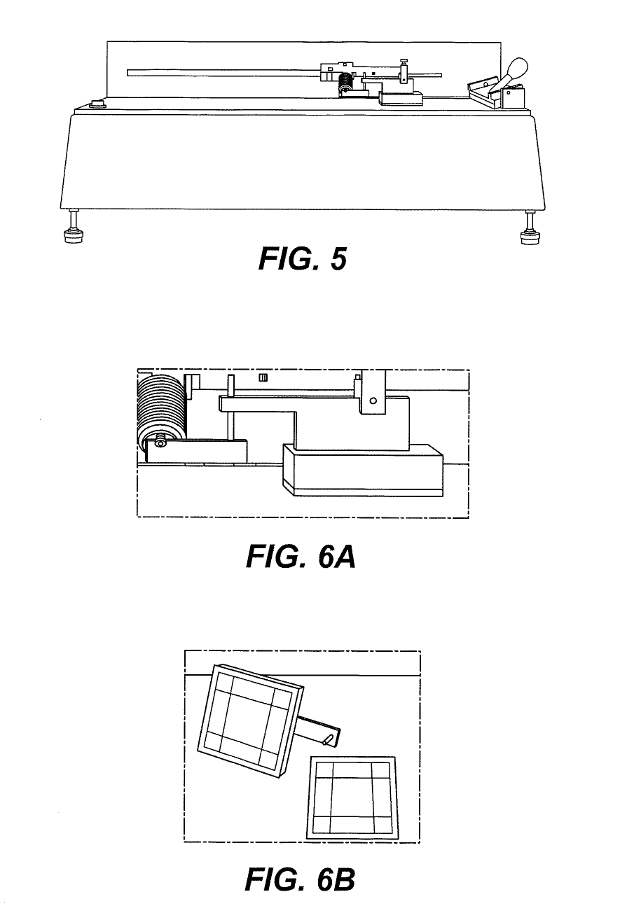

FIG. 5 is a drawing of a front perspective view of a friction tester used in the COF tests.

Sled Design

ASTM D 1894-08 “B” Sled (2.5″×2.5″, weight 200 g) as shown in FIGS. 6A & 6B is recommended for use in the friction testing per the ASTM. The “B” Sled (2.5″×2.5″, weight 200 g) is displayed which is recommended for use with the ASTM D 1984-08 standard. FIG. 6A illustrates the sled as inserted into the force gage of the friction tester and FIG. 6B illustrates the attachment of a sample onto the bottom of the sled using double-sided tape.

Initial friction testing was performed using this sled. However, a sled redesign was completed, after determining that the “B” sled design did not mimic the area in contact with the test surface during catheter usage. The lumen diameters of the aorta and femoral artery are around 25-30 mm (0.98″-1.18″) and 8-9 mm (0.31″-0.35″), respectively. Therefore, a new conformal sled with surface contact area that mimics that of the catheter to the lumen was developed. In addition to the surface area consideration, a rounded sled configuration was designed to replace the flat “B” sled. The design of the new sled is shown in FIGS. 7A, 7B, and 7C.

ASTM Modification—Design of ContraForm Sled

A new sled results in the amount of sample in contact with the test bed decreasing in comparison with the standard ASTM. As a result, a new sample size of 0.5″×2.5″ was selected. The ContraForm sled allows for a significantly smaller contact area of 0.5 in.×2.5 in. instead of 2.5″×2.5″ for the traditional sled. This is preferred as the contact area covered by the ContraForm sled more accurately mimics that of the catheter contact in vivo.

The samples (N=20) were tested on the friction tester at a temperature of 37° C. using the ContraForm sled. Each sample was run once. The test was performed on a 0.005″ thick PTFE test bed. A sample was affixed onto the bottom of the sled using double-sided tape and tested on the PTFE test bed through deionized (DI) water. A summary of the ASTM procedure modifications are listed below.

TABLE 7
Procedural Modifications from the ASTM D 1984-08

| Per ASTM D 1894-08 | Modification |
|---|---|
| Nothing regarding sterility | Sterile Samples |
| Traditional 200 g “B” sled | Newly Designed ContraForm |
| | Sled |
| Room temperature | 37° C. |
| Aluminum bare metal test bed | 0.005″ PTFE test bed |
| Non specified liquid media | DI water on test bed |

Fluoro-Additive Variations

In the above exemplary compositions, another fluoro additive in submicron mean particle size powder form can be substituted for the PTFE. Examples include FEP (Fluorinated ethylene propylene is a perfluoroalkoxy polymer resin (PFA)), eFEP from Daikin Industries, ETFE (Ethylene-co-tetrafluoroethylene, which is a copolymer of polyethylene and PTFE.

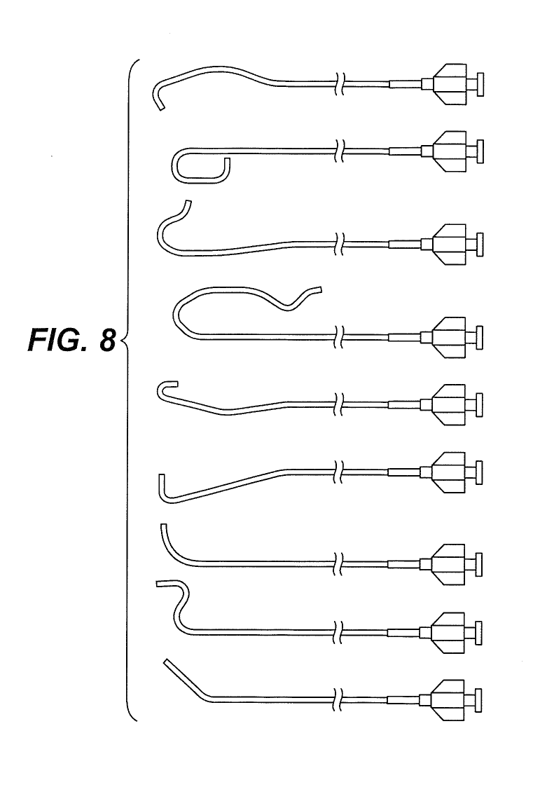

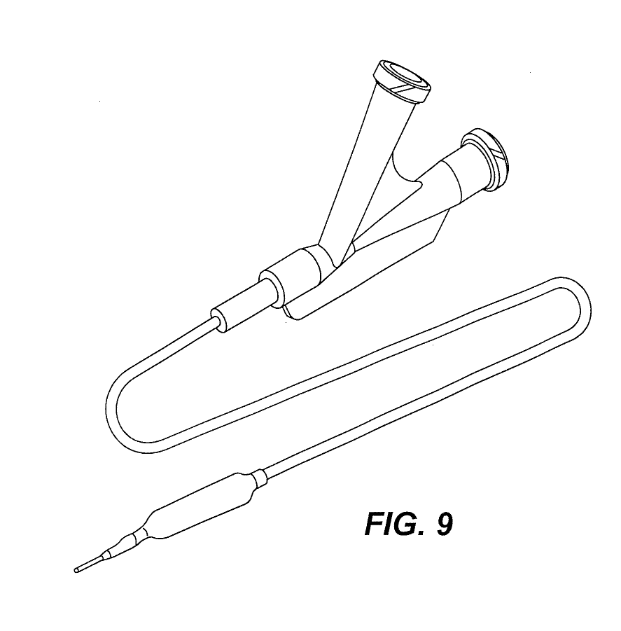

FIGS. 8 & 9 are medical devices which could include medical, intralumenal tubing made of this composition. FIG. 8 illustrates several guiding catheters of standard shapes, and FIG. 9 illustrates an angioplasty catheter, of which the inner tubular member (aka guide wire member, not shown, but running within the outer tubular member for at a length of the outer tubular member) or outer tubular member could be extruded of this composition.

In addition to guide catheters, other applications include inner coaxial bodies, outer coaxial bodies or outer members for interventional products. In addition, other products for which lubricity is critical for safe and effective function are potential applications of this extruded compound.

Aspects of the present invention have been described herein with reference to certain exemplary or preferred embodiments. These embodiments are offered as merely illustrative, not limiting, of the scope of the present invention. Certain alterations or modifications possible include the substitution of selected features from one embodiment to another, the combination of selected features from more than one embodiment, and the elimination of certain features of described embodiments. Other alterations or modifications may be apparent to those skilled in the art in light of instant disclosure without departing from the spirit or scope of the present invention, which is defined solely with reference to the following appended claims.

1. A composition of material comprising:
a poly(tetrafluoroethylene) (“PTFE”) powder by weight of the composition in the range of 1 to 25 percent;
and a polyamide in which the PTFE powder is dispersed; and
wherein the percentage of polyamide and the percentage of PTFE powder equals one hundred percent.

2. The composition of claim 1, wherein polyamide is a blend of homopolymer and co-polymers.

3. The composition of claim 2, wherein the blend includes nylon 12 homopolymer.

4. The composition of claim 2, wherein the blend include a polyether block amide.

5. The composition of claim 1, wherein the percentage of polyamide in the composition results from a percentage of nylon 12 homopolymer and the remaining percentage of a polyether block amide.

6. The composition of claim 5, wherein the polyether block amide has a lower durometer than the nylon 12 homopolymer.

7. The composition of claim 1, wherein the PTFE powder has a mean particle size in the range of 200 to 700 nanometers.

8. The composition of claim 1, further comprising a hydrophilic lubricant, wherein the percentage of polyamide is decreased the same percentage as the percentage of added hydrophilic lubricant.

9-16. (canceled)

17. A composition of material comprising:
approximately ninety percent by weight of nylon having a Shore-hardness measurement of 68D; and
approximately ten percent by weight of poly(tetrafluoroethylene) (“PTFE”) powder;
wherein the percentage of nylon and the percentage of PTFE powder equals one hundred percent.

18. The composition of claim 17, wherein nylon is a blend of nylon.

19. The composition of claim 17, wherein the blend of nylon includes nylon 12.

20. A guiding catheter comprising:
medical intralumenal tubing extruded from a compounded composition, wherein the composition comprises
a poly(tetrafluoroethylene) (“PTFE”) powder by weight of the composition in the range of 1 to 25 percent; and
a polyamide in which the PTFE powder is dispersed;
wherein the percentage of polyamide and the percentage of PTFE powder equals one hundred percent of any of the preceding claims.

21. The guiding catheter of claim 20, wherein polyamide is a blend of homopolymer and co-polymers.

22. The guiding catheter of claim 21, wherein the blend includes nylon 12 homopolymer.

23. The guiding catheter of claim 21, wherein the blend include a polyether block amide.

24. The guiding catheter of claim 20, wherein the percentage of polyamide in the composition results from a percentage of nylon 12 homopolymer and the remaining percentage of a polyether block amide.

25. The guiding catheter of claim 24, wherein the polyether block amide has a lower durometer than the nylon 12 homopolymer.

26. The guiding catheter of claim 20, wherein the PTFE powder has a mean particle size in the range of 200 to 700 nanometers.

27. The guiding catheter of claim 20, further comprising a hydrophilic lubricant, wherein the percentage of polyamide is decreased the same percentage as the percentage of added hydrophilic lubricant.
